(19) United States
(12) Patent Application Publication
(10) Pub. No.: US 2022/0096262 A1
(43) Pub. Date: Mar. 31, 2022

(54) ADVANCED ILEOSTOMY SUPPORT SYSTEM

(71) Applicant: The Curators of the University of Missouri, Columbia, MO (US)

(72) Inventor: James H. AUSTIN, Columbia, MO (US)

(21) Appl. No.: 17/490,163

(22) Filed: Sep. 30, 2021

Related U.S. Application Data
(60) Provisional application No. 63/085,520, filed on Sep. 30, 2020.

(51) Int. Cl. A61F 5/443; A61K 31/202; A61K 36/9066; A61K 31/353; A61F 5/449

(52) U.S. Cl. CPC A61F 5/443; A61K 31/202; A61F 5/449; A61K 31/353; A61K 36/9066

(57) ABSTRACT

An ileostomy stoma pouch support system includes a pouch coupled to an adhesive barrier attachable to a wearer around an ileal conduit, a barrier ring positionable around the ileal conduit and within the barrier such that the barrier ring is forming a seal with the pouch such that waste can enter the pouch, at least one barrier extender couplable to the barrier and extending a full circumference of the barrier, a washer placeable above the barrier and the at least one barrier extender such that the washer has at least some overlap with the barrier or at least one barrier extender through the full circumference of the barrier, and a cummerbund style belt adapted and configured to apply downward pressure on the barrier and the at least one barrier extender to hold the barrier and the at least one barrier extender against the skin of the wearer.

### CROSS-REFERENCE TO RELATED APPLICATIONS

This application claims the benefit of U.S. Provisional Application No. 63/085,520, filed Sep. 30, 2020, which is hereby incorporated by reference herein in its entirety.

### FIELD

The present teachings relate to ileostomy procedures, and more particularly an ileostomy stoma pouch support system.

### BACKGROUND

The statements in this section merely provide background information related to the present disclosure and may not constitute prior art.

Typical ileostomy stoma pouch support systems (e.g., ileostomy systems) use a slender belt to support the system when worn. A slender “belt” is basically a strap with a hook at each end that doesn't keep the stoma pouch secure from leakage because the abdomen around most patients' stoma is not a flat plain, but rather has hills and valleys that are not only irregular but also moveable. Moreover, the pouch is held under tension only by two hooks on either side. Ileostomy systems use barriers to attach to a wearer and seek to prevent leaks. The word, “barrier,” should alert the pouch wearer to the fact that it is only a temporary obstacle. Urine soon undermines one edge of this supposed “barrier.” After only four days or so of increasing social insecurity the pouch “springs a leak” and must be changed.

### SUMMARY

The above undesirable issues of known Ileostomy support systems can be overcome utilizing the Ileostomy support system and methods of the present disclosure. For example, in various instances five structural steps can be implemented toward a solution: 1) Replace the narrow 1⅛ inch belt of typical systems with a broad 8 inch band. This “cummerbund” style wrap supports the whole large region around all sides of the stoma. However, the pad inside this band is both too shallow and too diffuse to bring effective compression throughout 360 degrees. Therefore, 2) build several silicone rings into a larger, thicker washer or use a single washer. The washer is placed on top of the ileostomy system barrier so that downward pressure can be applied. Each silicone ring is 2 mm thick. Its hole is 2 and ¾ inch in diameter. Two to four of these layers can be tied together to suit each individual patient's local needs for peristomal compression. The result is a firm, flexible, force-vector of localized top-down-pressure. This washer acts to seal the vulnerable inner edge of the “barrier” underneath. Its compression now occurs throughout the full circumference of 360 degrees, not just at those two former side-pull sites—at 90° and 180°—that only exert tension in opposite directions. This uniform down-compression keeps the barrier's sticky side adherent to the skin directly beneath. 3) Apply three C-shaped, sticky barrier extenders to completely encircle the outer, non-sticky surface of this “barrier” beginning close to its innermost margin. 4) Apply a fourth C-shaped, sticky barrier extender, because this fourth C-shaped sticky extender acts like a kind of extra “roof” to stiffen and reinforce that most vulnerable segment of the “barrier.” The fourth C-shaped barrier extender is applied over a region that has been most undermined in the past and that has caused major leaks. 5) Apply one-half of a fifth C-shaped extender that will help further stabilize the same local segment of the adjacent silicone washer. This half-extender serves, like a localized “bridge,” to further press down on that washer's outer edge so that it keeps compressing that previously most leakable zone of the “barrier.”

This summary is provided merely for purposes of summarizing various example embodiments of the present disclosure so as to provide a basic understanding of various aspects of the teachings herein. Various embodiments, aspects, and advantages will become apparent from the following detailed description taken in conjunction with the accompanying drawings which illustrate, by way of example, the principles of the described embodiments. Accordingly, it should be understood that the description and specific examples set forth herein are intended for purposes of illustration only and are not intended to limit the scope of the present teachings.

### DRAWINGS

The drawings described herein are not to scale, are for illustration purposes only, and are not intended to limit the scope of the present teachings in any way.

FIG. 1 is an exemplary illustration showing the disadvantages of an ordinary slender hook belt used in known side-pull Ileostomy support systems.

FIG. 2 is an exemplary illustration showing the advantages of multiple layers of localized compression, in accordance with various embodiments of the present disclosure. The stoma shown in both figures actually extends further up through the hole in the convex cera portion than is shown in both illustrations.

FIG. 3 is an exemplary illustration showing the ordinary slender hook belt of FIG. 1.

FIG. 4 is an exemplary schematic illustration showing the wearer side of the ileostomy system of the present disclosure.

FIG. 5 is exemplary schematic illustration showing the side facing away from the user of the ileostomy system of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements. Additionally, the embodiments disclosed below are not intended to be exhaustive or to limit the invention to the precise forms disclosed in the following detailed description. Rather, the embodiments are chosen and described so that others skilled in the art can utilize their teachings. As well, it should be understood that the drawings are intended to illustrate and plainly disclose presently envisioned embodiments to one of skill in the art, but are not intended to be manufacturing level drawings or renditions of final products and may include simplified conceptual views to facilitate understanding or explanation. As well, the relative size and arrangement of the components may differ from that shown and still operate within the spirit of the invention.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims.

Unless otherwise defined, all technical and scientific terms used herein have the same meaning as commonly understood by one of ordinary skill in the art to which this disclosure belongs. The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps can be employed.

When an element, object, device, apparatus, component, region or section, etc., is referred to as being “on”, “engaged to or with”, “connected to or with”, or “coupled to or with” another element, object, device, apparatus, component, region or section, etc., it can be directly on, engaged, connected or coupled to or with the other element, object, device, apparatus, component, region or section, etc., or intervening elements, objects, devices, apparatuses, components, regions or sections, etc., can be present. In contrast, when an element, object, device, apparatus, component, region or section, etc., is referred to as being “directly on”, “directly engaged to”, “directly connected to”, or “directly coupled to” another element, object, device, apparatus, component, region or section, etc., there may be no intervening elements, objects, devices, apparatuses, components, regions or sections, etc., present. Other words used to describe the relationship between elements, objects, devices, apparatuses, components, regions or sections, etc., should be interpreted in a like fashion (e.g., “between” versus “directly between”, “adjacent” versus “directly adjacent”, etc.).

As used herein the phrase “operably connected to” will be understood to mean two or more elements, objects, devices, apparatuses, components, etc., that are directly or indirectly connected to each other in an operational and/or cooperative manner such that operation or function of at least one of the elements, objects, devices, apparatuses, components, etc., imparts are causes operation or function of at least one other of the elements, objects, devices, apparatuses, components, etc. Such imparting or causing of operation or function can be unilateral or bilateral.

As used herein, the term “and/or” includes any and all combinations of one or more of the associated listed items. For example, A and/or B includes A alone, or B alone, or both A and B.

Although the terms first, second, third, etc. can be used herein to describe various elements, objects, devices, apparatuses, components, regions or sections, etc., these elements, objects, devices, apparatuses, components, regions or sections, etc., should not be limited by these terms. These terms may be used only to distinguish one element, object, device, apparatus, component, region or section, etc., from another element, object, device, apparatus, component, region or section, etc., and do not necessarily imply a sequence or order unless clearly indicated by the context.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because many varying and different embodiments may be made within the scope of the concept(s) taught herein, and because many modifications may be made in the embodiments described herein, it is to be understood that the details herein are to be interpreted as illustrative and non-limiting.

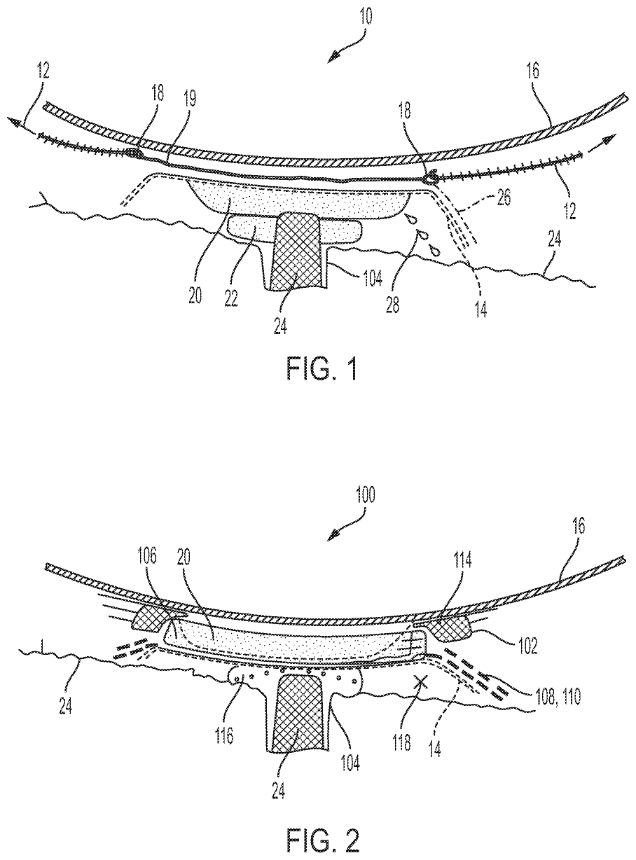

Referring generally to FIGS. 1-5, an existing side-pull ileostomy system 10 can be modified to achieve the benefits described herein resulting in an improved system 100. That old system 10 includes a thin belt 12 to apply tension and hold the barrier 14 and pouch 16 in place. The belt 12 attaches to the remainder of the system 10 with hooks or other fasteners that connect to a loop 18 and/or other fastening structure 18 (e.g., a support ring 19). This in turn holds a convex portion 20 of the pouch 16 in place along with a ring barrier 22 on the wearer's skin 24 and about an ileal conduit or stump 24 of the wearer. Barrier extenders 26 can be used to extend the existing barrier 14. Leaks 28 are prone to occur after several days.

Referring again to FIGS. 1-5, in general, an existing narrow 1 and ⅛ inch belt 12 is replaced with a broad 8 inch band 102. This “cummerbund” style wrap (e.g., cummerbund style belt) 102 supports the whole large region on all sides 104 around the stoma 24. However, the broad 5 and ¾ inch pad inside this band is both too shallow and too diffuse to bring effective compression throughout 360 degrees. Therefore, the system 100 also includes a 3 and ¾ inch washer 106 to act as the critical seal.

A wearer can build several silicone rings into a larger, thicker washer 106. For example, each silicone ring is 2 mm thick. Its hole is 2 and ¾ inch in diameter. Two to four of these layers can be tied together to suit each individual patient's local needs for peristomal compression. The result is a firm, flexible, force-vector of localized down-pressure. This washer 106 acts to seal the vulnerable inner edge of the “barrier” 14 underneath. Its compression now occurs throughout the full circumference of 360 degrees, not just at those two former sites—at 90° and 180°—that only exert a side-pull in opposite directions. This uniform down-compression now keeps the barrier's 14 sticky side adherent to the skin 24 directly beneath.

The system 100 also includes three C-shaped, sticky barrier extenders 108 applied to completely encircle the outer, non-sticky surface of this “barrier” (e.g., the barrier 14 of an existing pouch system 10) beginning close to its innermost margin.

The system 100 further includes a fourth C-shaped, sticky barrier extender 108 applied in a region prone to leaks. Because this fourth C-shaped sticky barrier extender 110 acts like a kind of extra “roof”, it stiffens and reinforces that most vulnerable segment of the “barrier.” This particular region is reinforced due to its being the one most undermined in the past and then causing major leaks. The system 100 does not include loops, tabs, or other similar fasteners (e.g., the lack of such a fastener is illustrated in region 114). The system further holds in place a ceramide containing barrier ring 116 around the ileal conduit or stump 24. As a result, leaks are reduced (e.g., a lack of leaks is illustrated in region 118).

The system 100 also includes an applied one-half of a fifth C-shaped extender 112 that will help further stabilize the same local segment of the adjacent silicone washer 106. This half-extender 112 serves, like a localized “bridge,” to further press down on that washer's 106 outer edge so that it keeps compressing that previously most leakable zone of the “barrier” 14 (e.g., as shown in FIG. 4).

Generally, the foregoing provides a brief description of a method of updating an existing side-pull system 10 to an improved, focused compression system 100. Hollister® products or the other products mentioned are exemplary products that can be used when changing the stoma pouch 16. However, these products are exemplary only and the systems and methods described herein could be used with other improved components or products. The first change when moving away from the old system 10 to the advanced system 100 is to carefully cut off the two plastic tabs 18 that stick out from either side of the Hollister® 8412 ¾ inch hole Cera Plus Fabric Pouch 16. After this, its large, temporary clear plastic cover is removed. Then, the ceramide-containing barrier ring is stripped of its coverings. This ring is the Hollister® 8815—⅝ inch hole Adapt Cera Slim Ring 116. Its sticky surface is now being pressed against the convex ceramide surface of the pouch 20, making sure that the two holes are perfectly aligned. Next, tweezers can help to peel off the long temporary strips that cover the underside of the pouch 16. The next step involves Hollister® 79402. This is a C-shaped Barrier extender 108 that is sticky on only one side. Peel the covering from both middle sections back to ¾ of an inch from the ends. Carefully apply three of these to enclose the whole circumference of the barrier 14. Follow with an additional one 110, and a final half one 112, to cover the zone most vulnerable to leakage.

Next, wads of strong bathroom tissue can be used (e.g., 5 dry; 4 moistened with warm water). The physical cleansing of the skin is given priority. Strong, disposable tissue does not easily disintegrate into residual fragments, nor is it as likely to be contaminated with bacteria as is an ordinary fabric washcloth. Gently scrub the skin repeatedly. Dry thoroughly. Reserve an extra large dry clump to blot spurts of urine. Introduce mild (chemical) soap solution as indicated if any signs of infection. Subsequently, curved surgical scissors can be utilized (e.g., 5 and ½ inch and 3 and ¼ inch). First use the large scissors to cut off the two plastic loops 18 at each end of the plastic ring 19. Later, the large curved blade scissor can be used to gently scrape off Cera residue and remove lose skin fragments. Use the small scissors to snip off recurrent fine hairs. Thereafter, the silicone canning rings or washer(s) 106 are bound together into one large functional unit, e.g., using four double ties of dental floss, spaced 90° apart. Three or four rings, tied together, make a thicker “washer” 106 and can provide a better seal than only two rings. Finally, a cummerbund style belt 102 is utilized. This 8 inch wide belt with gripping features stabilizes the whole lower abdomen. Its uniform, elastic compression effect also helps compensate for an actual or impending peristomal hernia.

Additional details during pouch changes. First, empty the old pouch 16 completely before beginning. Slowly peel off the old “barrier” 14 and its extenders 26, starting at top. Changing pouches when first arising in the a.m. helps reduce the tendency to have urine spurts. Next, blot all urine from inside edges of the pouch 16. Gently scrub and carefully clean skin surface until moist tissue becomes colorless. Dry thoroughly with dry tissue. Avoid contact with the red ileal stump 24 in center. Next, having already removed the protective covering from one side of the replacement ceramide containing barrier ring 116, center the hole of this ring 116 to fit over the protruding stump 24 (e.g., as shown in FIG. 2). Gently press on the outer (still covered) side to help this ring 116 adhere to the skin. Then peel off that outer covering of the ring. Then, apply the convex portion 20 of the new pouch 16 to match the hole in the newly applied ceramide containing barrier ring 116. This step can be reversed by first applying the ring to the pouch 20. Using repeated, warm, finger pressure, smooth the circumference of the sticky “barrier” ring 116 so that it covers the skin surface evenly. Next, gather together the edges of the pouch 16 in order to clearly visualize the inner edges of this “barrier” 14 hidden beneath. Next, carefully apply 3 barrier extenders 108, and then the fourth 108 (not shown in FIG. 4), starting on the right side. Leave no gaps or folds. Cover not only up to, but just under, the innermost circumference of the clear plastic protruding at the barrier edge. Then, slide the tied silicone ring-washer 106 around the folded pouch 16. This thick washer unit 106 now focuses compression on the innermost portions of the barrier extenders 108, 110 beneath. Apply one-half of the fifth extender 112 to bond the washer's 106 edge to the most vulnerable zone. Finally, again, fold the edges of the pouch 16, this time through the open hole in the cummerbund style belt 102 (e.g., support band). The inner margins of this hole now push down on the top of the silicone washer 106. Sometimes, after a week, the circular opening of this support band 102 may widen. When such an enlargement no longer compresses the underlying silicone washer 106, change into a fresh support band 102 that has shrunken into a smaller opening closer to 2 and ¾ inch.

Use of two Hollister® products—the Hollister® Adapt Cera Slim Ring (8815) and the Cera Plus Convex portion of the larger pouch (8412)—provide major technical advances. This word, Cera, refers to ceramide. Ceramide is the major new biochemical constituent of these ostomy projects. The ceramide molecule links a base, sphingosine, with a fatty acid. These ceramide esters are natural constituents of normal skin and brain. The goal is to make the artificial hydrocolloid ring and convex pouch so closely resemble the patient's own skin that neither product excites a foreign body inflammatory reaction.

Wearers of the system 100 should be guided by the ostomy nurses. They are wary of any untested procedures that depart from the long-held, industry standard, “4+ day change” regimen for skin hygiene. Patients and nurses must always remain alert to scale back, either at the first evidence of infection or at the first hint of discoloration under the “barrier” that signals a leak is in progress. The intervals between pouch changes are determined by experimental testing. The intervals should only increase gradually, when verified by the prior patterns of the test results.

A wearer can be strongly motivated by the needs to achieve the much lower costs, greater convenience, and leak security associated with longer intervals between pouch changes provided by the system 100 and associated method. Each wearer must understand why it is essential to stay well-hydrated and to empty the pouch 16 frequently. Each must have the cognitive capacity to consistently follow the requisite directions. Vision must not be obstructed by gross abdominal obesity. Because wearers can turn over while sleeping, it is advised that wearers wear the cummerbund style belt night and day, except when bathing. Ceramide containing barrier rings are a dissolvable hydrocolloid. They can last longer inside the cooler temperature of a well-ventilated pouch.

During application of the system 100, fine-spray and wipe off all working surfaces and scissors using Chlorox Cleaner-Bleach (Sodium Hypochlorite). Thoroughly wash hands with soap and water. After each use of the hand sanitizer (70% ethyl alcohol), dry fingers before touching pouch.

At least daily inspections of the system 100 should be performed. Local discoloration under the pouch's 16 fabric “barrier 14” or its C-shaped barrier extenders 108, 110 is a warning signal that the pouch 16 will need changing. Use the window in the front of the fabric pouch 16 to inspect the peri stomal 24 region for any sign of infection, mold, and/or bleeding. Avoid repeated contact between the pouch 16 and clothing (e.g., a belt is worn below the pouch, not over it). The pouch 16 can be reinforced locally with tape if its fabric becomes frayed.

Wearers should avoid extreme movements that distort the pouch 16. Wearers should also avoid bending over at the waist to pick up anything. Instead, wearers should bend at both knees.

Further ancillary dietary measures should be followed to prevent breakdown of the skin and to increase the longevity of the stoma pouch attachment and the time between changing of the stoma pouch. For example, such ancillary dietary measures can include a regimen of anti-inflammatory measures including an omega-3 fatty acids supplement containing both eicosapentaenoic acid (EPA) and docosahexaenoic acid (DHA). For example, the supplement can be a one time daily 1200 mg capsule of fish oil containing 360 mg of omega-3, both DHA and EPA. The dietary measures can include one 450 mg capsule taken four times a day of Algal DHA. This supplement provides anti-inflammatory benefits which reduce skin irritation at the site where the ileostomy system is worn and decreases the need to change the ileostomy system. The dietary measures can include supplements that provide high blood levels after absorption. For example, this supplement can be a daily 500 mg Turmeric Extract BCM-95® Curcumin Elite. Curcumin (e.g., turmeric extract) provides an unexpected benefit in that it increases the endogenous synthesis of ceramide. Ceramides have the benefit of improving skin health and reducing skin irritation. This can prolong the useful life of each ileostomy system. The dietary measures can include a quercetin supplement that can also include phytosome. For example, the supplement is a once daily 29 mg Bio-Quercetin®. Quercetin provides a benefit in that it aids in the creation of antibacterial films that reduce inflammation and aid skin integrity. The wearer can also adopt a low or reduced carbohydrate diet to combat inflammation.

Referring now to FIGS. 2-5, a method of using an ileostomy support system 100 is described in further detail as follows. This advanced method solves several problems that cause leakage in existing ileostomy support systems 10.

The main problem is that the existing simple strap systems have to be changed every four or five days because they leak unpredictably in socially compromising situations. If you change them only every four days or so, by the end of a year you've got many hundreds of dollars involved just in your equipment. If the techniques described herein are followed, unexpected leaks are postponed for multiple days. On numerous earlier occasions, the intervals between elective changes can then be extended up to 16 days. More recently, elective changes every nine or ten days are being observed. Whether such variations represent chemical differences among different lots of the constituents of the hydrocolloid ring, or biochemical changes in the wearer, is not clear at this time.

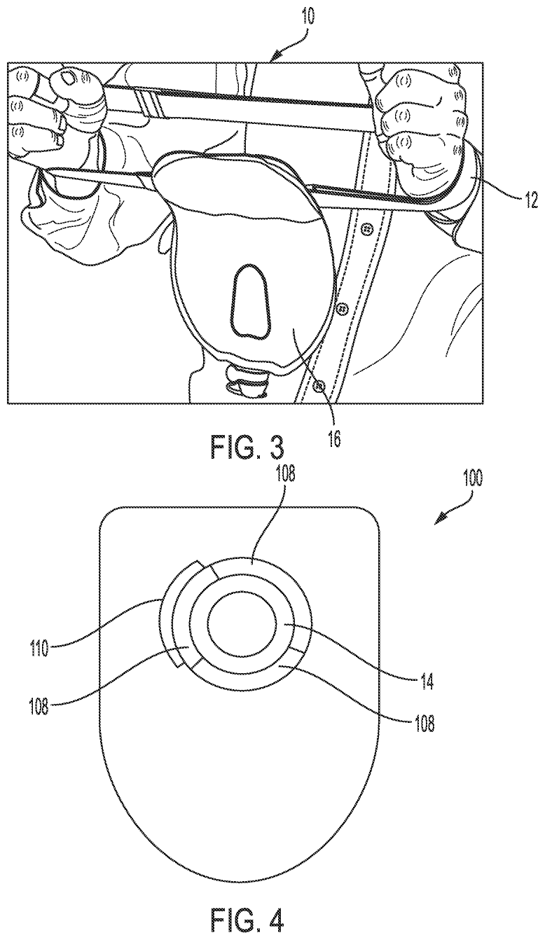

A fundamental problem with existing systems and methods of ileostomy support and wearing is that such systems and methods depend on tension being applied only at the end on each side (e.g., from a hooked belt 12 pulling laterally). This means tension is coming in only at nine o'clock and three o'clock when the pouch 16 is worn (e.g., along, and parallel to, a wearer's waist). This is shown in FIG. 3. In existing systems, a slender belt 12 is attached to the pouch with hooks. These engage with tabs 18 on the pouch itself 16. The strap-belt 12 does not extend across the pouch—being attached at the sides. As a result, no direct force or pressure being applied perpendicular to the pouch and towards the wearer's body. With such existing systems, the pouch can be easily displaced up and down by a simple force pulling in various directions. This movement of the pouch can result in the development of leaks through the circular barrier 14 portion of the pouch after it becomes detached from the wearer's skin 24. For example, a leak 28 can frequently occur somewhere around the seven o'clock position (when the spout of the pouch points down at the six o'clock position). Such a leak can result, for example, whenever movement of the pouch delaminates that barrier from the skin of the wearer. This situation is made worse by most wearers' naturally uneven skin topography.

Of course, and as previously explained, the sticky barrier 14 is not a permanent and/or complete barrier to leaks. A barrier is not a tight wall. It is a leaky barrier. A strap and two hooks constitute short life equipment, not equipment that is designed to be longer lasting.

The alternative system 100 and method described herein, and in more detail in the following, improves upon existing barriers both to better prevent leaks and to extend the time between elective ileostomy pouch changes. These systems and methods acknowledge that leaks can occur, for no barrier is completely leak proof. Moreover, they improve upon the construction of previous systems by applying a top-down compression to the pouch. This directly presses the barrier components against the wearer's skin. It does not rely on indirect lateral tension on the sides to stretch the barrier against the skin of the wearer, as in previous systems.

Previous systems were originally designed on the premise that our abdomen around the ileostomy is flat. However, abdomens come with hills, valleys, bumps, lumps, and other topographical changes and features. Furthermore, different muscles and fat layers lie underneath the abdomen at the site of the ileostomy. Different wearers exert different torsions as they move from side to side and up and down. Therefore, we need a flexible system with more inherent stability. This system 100 can be produced by modifying and adding to existing systems. For example, a Hollister® ileostomy pouch product number 8412, as shown in FIGS. 1 and 3 as 10, can be modified. The systems and methods described herein can be performed by modifying or otherwise using any suitably appropriate ileostomy pouch, the foregoing be exemplary only.

Initially, the sticky barrier 14 is covered over with a plastic film so that it doesn't stick elsewhere prior to application. On opposing sides of the opening to accommodate the ileal conduit 24 (e.g., ileal stump) and positioned between the barrier and the pouch itself are two clear plastic tabs with ring holes 18. These tabs (e.g., positioned at three o'clock and nine o'clock when looking at the pouch as worn) are removed. The tabs can be removed, for example, by carefully cutting the tabs 18 from the pouch 16. The tabs are not necessary in view of the wide support belt used in the systems and methods described herein. Moreover, removal of the tabs also improves performance of the pouch and system described herein. When the tabs 18 are removed, they no longer interfere in the application of compression perpendicular to the pouch and towards the wearer when the pouch is worn. Were tabs or other components left in place, they would interfere with the evenness, for example, of the top-down compression.

As shown in FIG. 4, the wearer applies three barrier extenders 108 to the existing barrier 14 of the pouch 16. The barrier extenders can, for example, be Hollister® part number 79402 or some other suitable ileostomy barrier extender. In one embodiment, the barrier extenders 108 are added underneath the existing barrier. In alternative embodiments, the barrier extenders are added on top of the existing barrier relative to the wearer's skin when worn. Each barrier extender is slightly longer than a third of a circle. Therefore, the barrier extenders overlap the existing circular barrier and increase the original barrier's circumference to provide further 360 degree coverage.

Also as not shown in FIG. 4, a fourth barrier extender 108 can be added. This fourth barrier extender 108 is added for further coverage of that particular region in which a wearer personally experiences common leaks. The fourth barrier extender 108 can be positioned in any position. Experimentally, it has been found beneficial to place the fourth barrier extender at the seven o'clock position. In some embodiments, this fourth barrier extender is optional.

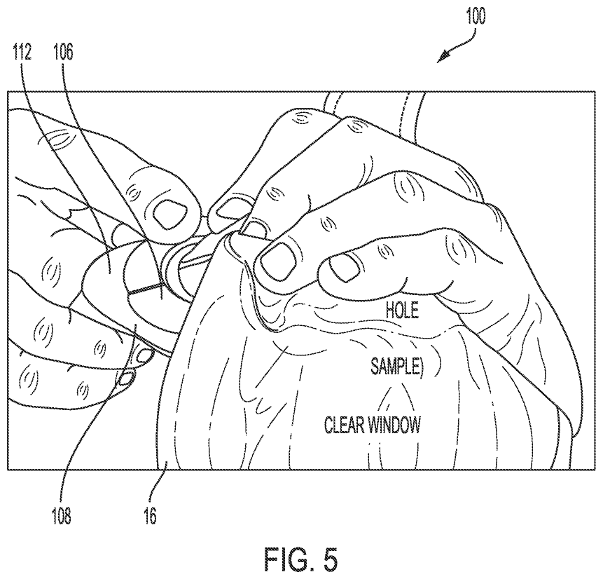

As shown in FIG. 5, a washer 106 is added on top of the combined barriers relative to the wearer's skin. The washer acts as a seal. The washer 106 is silicon in one embodiment, but it can be other suitable biocompatible material. As previously explained herein, the washer can be constructed of a plurality of thin washers coupled together. The number of washers can be adjusted, separately or in conjunction with the length and fit of the cummerbund style belt 102. The goal is to apply the requisite amount of downward compression to the barriers so as to securely hold the pouch against the wearer's skin. When several individual washers are used, they are bound together using any suitable technique (e.g., adhesive, string, dental floss, or the like). The washers can be secured together at three or four locations to provide for a unified washer assembly. Sufficient space between the ties is reserved for the fifth (½ c-shaped) barrier extender to be attached. In one embodiment, the individual washers are between 1/16 to ⅛ inch thick. Their outer diameter is 3 and ¾ inches, and their inner diameter is 2 and ½ inches. For example, the individual washers can be Harold Import Co® canning rings, part number 9924. In such cases, a tab (not shown) on each washer is cut off. Notice that the outer diameter of the washer 106 is greater than the diameter of the opening in the cummerbund style belt 102 through which the pouch 16 extends. This means that the cummerbund style belt applies appropriate compression on the underlying washer when worn. This compression is applied perpendicular to the pouch and toward the wearer. This enables the barrier and barrier extenders to remain directly pressed down against the skin of the wearer. These levels of compression increase the effectiveness of the barrier (e.g., the combined barrier 14 and barrier extenders 108, 112). The result helps prevent leaks, in contrast with existing side-pull systems which do not apply such a top-down force vector.

Still referring to FIG. 5, in order to secure the washer or washers to the barrier(s), a fifth (½ c-shaped) barrier extender 112 is added. This enables the washer(s) 106 to adhere to the barrier(s) beneath. This fifth barrier extender is half the size of the first through fourth barrier extenders. In alternative embodiments, adhesive tape or other fasteners can be used to secure the washer(s) to the barrier(s) or to another component of the pouch or pouch system. Advantageously, the fifth barrier extender strengthens and stiffens the assembly of barrier(s) and washer(s) which reduces movement of the washer(s) and barrier(s) relative to one another when the wearer moves substantially. The top down pressure applied through the washer(s) puts pressure on the barrier(s) helping to prevent leaking (e.g., at any spot where leaks typically develop for a wearer) by aiding in the connection between the barrier(s) and the wearer's skin (e.g., by reducing movement of the barrier(s) relative to the skin, countering or balancing the result counters or balances delamination forces, or the like).

As shown in FIG. 2, the wearer applies a ceramide containing barrier ring 116 that adheres on one side to the convex Cera 20 of the pouch 16 and to the peristomal skin on its other side. This barrier ring 116 will extend around the stump 24 of the ileal conduit or stump 24. The ceramide containing barrier ring is held in place by the barrier and/or by the pouch itself and can be placed in a receptacle 20 of the pouch for receiving such rings. For example, the ceramide containing barrier ring can be Hollister® part number 8815, Cera Ring Slim Barrier Ring. Ceramides are molecules composed of two ingredients, one of them is a base and the other is a fatty acid. Ceramides are a normal vital ingredient in skin and assist in skin health. By maintaining healthy skin, the ileostomy system can be worn for a longer period of time (e.g., without needing to be changed because the skin is irritated). Ceramides can also reduce inflammation because the ceramide molecule does not act like an artificial foreign body and is not treated as such by the body. Ceramides are a normal biochemical substance and therefore the wearer is not going to have an inflammation response due to an artificial product next to the wearer's skin.

On preparing the ileostomy system 100 and performing the method described herein, the wearer adopts standard antiseptic procedures. These can include, for example, spraying the surface of the area chosen for the pouch change with Clorox® or other suitable disinfectant, and then wiping it off to dry; scrubbing the hands thoroughly with soap and water; applying a 70% ethyl alcohol disinfectant lotion at multiple times throughout, and being sure to dry off one's hands so that no alcohol or Clorox® comes in contact with any of the ostomy products.

Returning to FIG. 2 and the ceramide containing barrier ring 116. When exposed to liquid urine, the ceramide rings hydrocolloids and other constituents slowly disintegrate. Observed through the clear window of the pouch, this first looks like a loose aggregate of solubilized material around the pink stoma. It then becomes a light “white collar” around the stoma. This shape expands outward and inward. It later evolves into a wider and more compact “white ruff”. This inward expansion can encroach on the center of the stoma. This limiting factor dictates how soon the pouch will need to be changed. Accordingly, various modifications have been made in the volume of the ring in its constituents and in the rest of the procedure. Overexpansion and/or degradation of the ceramide containing barrier ring can also result in encroaching on the ileal stump and blocking the flow of urine. When this hydrocolloid ring is exposed to water it dissolves. The ceramide containing barrier ring starts as a disc, and as it deteriorates takes on a ruff-collar shape.

The above noted deterioration of the ceramide containing barrier ring 116 can be further mitigated by reducing the amount of heat to which the ceramide containing barrier is exposed. Heat (e.g., body heat or heat from urine entering the pouch) speeds degradation of the ceramide containing barrier ring. This effect of heat can be mitigated by, for example, slicing open the center of the hooded pouch flap covering the outside of the pouch 16. This enables room air or ambient air to cool the surface and to keep the pouch temperature somewhat below that of the warm urine coming out at abdominal and pelvic temperatures. In some embodiments, the wearer can store ceramide containing barrier rings in a cold location (e.g., in a refrigerator at for example four degrees Celsius for weeks) prior to use. The storage in a cold environment can help maintain the integrity of the ceramide containing ring before use. In such embodiments, the wearer wraps the box containing the ceramide containing barrier ring in a closed plastic bag to keep out moisture or otherwise protects the ceramide containing barrier ring while stored (e.g., using a plastic bag).

Returning to general skin care in support of ileostomy systems 100 and methods described herein. When cleaning the skin, extraordinary bathroom tissue is most appropriate, especially strong bathroom tissue (e.g., Charmin® Ultra Strong). The bathroom tissue should be of a type where its fibers don't break down easily when warm water is used to scrub the skin around the stoma. After repeated blotting and scrubbing lightly and gently, the result is a clean skin surface which is primed and ready to accommodate the pouch convexity which now has the ceramide containing barrier ring attached to it. The wearer now places the pouch 16 with its adherent ceramide containing barrier ring 116 precisely around the stump of the ileal conduit 24 (e.g., having first removed any protective layer from the barrier and/or barrier extenders so that their adhesive layer is exposed). These adhesive layers of the barrier 14 and of the barrier extenders 108, 110 (e.g., the three barrier extenders positioned around 360 degrees and the fourth barrier extender positioned over a location prone to leaks) will further attach the pouch to the wearer's skin 24. The washer 116 or plurality of washers (e.g., washer assembly) is situated on top of the barrier and barrier extenders.

Next, the cummerbund style belt 102 is added, becoming the outer layer of the top-down system. The length of this support wrap is chosen to be more than sufficient to extend around the wearer. Its width is, for example, between 8, 9, 10 or 12 inches. This particular style contains a through hole surrounded by a thin pad. Several of the next hole sizes now become critical. The cummerbund's 2 and ½ inch wide through hole can be adjusted slightly to accommodate the size of the pouch 16. This pouch extends out through the hole to the exterior of the cummerbund while the ileostomy system 100 is being worn. The diameter of the through hole is ¼ inch less than that of the flexible washer(s). This ensures that the cummerbund style of support belt can deliver compression on the washer(s) beneath it. The cummerbund style belt has a Velcro® fastener. This enables it to securely adjust its circumference and thus to maintain a reasonably consistent degree of compression on the washer(s) beneath it. This compression is then transmitted down through the washer(s), and the barrier, and the barrier extenders to the wearers skin. (Such an adjustable fastener could also be a hook and loop fastener, a strap, a small buckle, or something similar). In order to maintain a consistent degree of compression, from one change to the next, small dots of white-out correction fluid are used to mark the site of the adjustable leading edge of the overlapping Velcro® fastener. The interior side of the cummerbund style belt includes a plurality of gripping features. For example, these gripping features can be nubs, protrusions, hemispherical domes, or the like. The gripping features increase the adherence to the skin of the wearer of the cummerbund and reduce its slipping or displacement. In one embodiment, the cummerbund style belt is a Safe N Simple™ Support Belt.

As previously explained, the cummerbund style belt 102 replaces the typical inch and a half strap 12 that have hooks which only pull laterally. This is a major advantage over the typical strap. The elasticity of the cummerbund also helps support the abdomen and contributes to the dynamic flexibility of a belt system that is clearly going to move up and down, and from side to side. The cummerbund's degrees of compression can be varied empirically to arrive at a fit that is snug, rather than too tight or too loose.

One cosmetic disadvantage of the cummerbund style belt 102 is that it does add an extra layer to one's waist. However, one can cover the whole system 100 with an untucked shirt, or with a high-waisted skirt, or any number of other items of clothing that can conceal the fact that one is wearing an ileostomy pouch. For bedroom matters, the empty ileostomy pouch, minus the cummerbund, can be enclosed in a very thin wide, elastic fabric. This has two nests or small pouches, one on either side, into which the wide, pouch 16 can be placed.

The wearer continually monitors the ileostomy system to determine when it needs to be changed. For example, as the system is worn, the ceramide containing barrier ring 116 is exposed to heat and liquid and begins to deteriorate. The ceramide containing barrier ring changes from a tan color toward a white color. As the ceramide containing barrier ring dissolves and expands, it reforms as a little collar around both the inside and the outside of the ¾ inch hole within the convex ceramide surface of the pouch itself 20. The wearer monitors the deformation of the ceramide containing barrier ring to ensure that it doesn't intrude too far toward the center and interfere with the drainage. The ileostomy system is changed selectively as frequently as necessary, rather than because it has leaked precipitously.

In some embodiments, the wearer takes further ancillary steps to increase the interval of time between changes in the ileostomy system. The wearer takes an omega-3 fatty acid supplement containing both eicosapentaenoic acid (EPA) and docosahexaenoic acid (DHA). For example, the supplement can be a one time daily 1200 mg capsule of fish oil containing 360 mg of omega-3, both DHA and EPA. The supplement also includes one 450 mg capsule taken four times a day of Algal DHA. This supplement provides anti-inflammatory benefits which reduce skin irritation at the site where the ileostomy system is worn and decreases the need to change the ileostomy system. The dietary measures can include supplements that provide high blood levels after absorption. For example, this supplement can be a daily 500 mg Turmeric Extract BCM-95® Curcumin Elite. Curcumin (e.g., turmeric extract) provides an unexpected benefit in that it increases the endogenous synthesis of ceramide. As previously explained, ceramides have the benefit of improving skin health and reducing skin irritation. This can prolong the useful life of each ileostomy system. The dietary measures can include a quercetin supplement that can also include phytosome. For example, the supplement is a once daily 29 mg Bio-Quercetin®. Quercetin provides a benefit in that it aids in the creation of antibacterial films that reduce inflammation and aid skin integrity. This in turn reduces the frequency with which the ileostomy system needs to be changed. The wearer can also adopt a low or reduced carbohydrate diet to combat inflammation.

Typical ileostomy systems requiring changing as frequently as every four or five days, sometimes precipitously because they leak. In the past, it has been determined experimentally that the system and the method described herein have sometimes enabled the wearer to wear this ileostomy system for up to 16 days. Measure are currently under way to extend the average intervals.

The description herein is merely exemplary in nature and, thus, variations that do not depart from the gist of that which is described are intended to be within the scope of the teachings. Moreover, although the foregoing descriptions and the associated drawings describe example embodiments in the context of certain example combinations of elements and/or functions, it should be appreciated that different combinations of elements and/or functions can be provided by alternative embodiments without departing from the scope of the disclosure. Such variations and alternative combinations of elements and/or functions are not to be regarded as a departure from the spirit and scope of the teachings.

What is claimed is:

1. An ileostomy stoma pouch support system, said system generally comprising:
a pouch coupled to an adhesive barrier having an inner diameter such that the barrier can be attached to a wearer around an ileal conduit;
a barrier ring positionable around the ileal conduit and within the barrier such that the barrier ring forming a seal with the pouch so that urine can enter the pouch;
at least one barrier extender couplable to the barrier and extending a full circumference of the barrier;
a washer placeable above the barrier and the at least one barrier extender such that the washer has at least some overlap with the barrier or at least one barrier extender through the full circumference of the barrier; and
a cummerbund style belt adapted and configured to be worn by a wearer and support the system, the cummerbund style belt including a through hole with a diameter less than an outer diameter of the washer, the cummerbund style belt further adapted and configured such that, when worn, the pouch extends through the through hole and the washer does not extend through the through hole, the cummerbund style belt applying downward compression on the washer and towards the wearer's skin, he washer in turn applying downward pressure on the barrier and the at least one barrier extender to hold the barrier and the at least one barrier extender against the skin of the wearer throughout the full 360° circumference of the barrier.

2. A system in accordance with claim 1, wherein the at least one barrier extender comprises three barrier extenders adapted and configured to be coupled to the barrier to extend the full circumference of the barrier.

3. A system in accordance with claim 2, wherein the three barrier extenders are adapted and configured to be coupled to the barrier to expand the area couplable to a wearer's skin.

4. A system in accordance with claim 2, further comprising a fourth barrier extender, the fourth barrier extender couplable to the barrier.

5. A system in accordance with claim 4, wherein the fourth barrier extender is adapted and configured to be coupled to the barrier to expand the area couplable to a wearer's skin.

6. A system in accordance with claim 4, further comprising a fifth barrier extender, the fifth barrier extender adapted and configured to couple the washer to the barrier or the at least one barrier extender.

7. A system in accordance with claim 1 wherein the washer comprises a plurality of individual washers.

8. A system in accordance with claim 7 wherein the plurality of individual washers includes three or more individual washers.

9. A method for changing an ileostomy stoma pouch, said method comprising:
placing a barrier ring around an ileal conduit
attaching at least one barrier extender to a barrier of a pouch;
placing a washer above the barrier and the at least one barrier extender such that the washer has at least some overlap with the barrier or at least one barrier extender through the full circumference of the barrier and extending the pouch through the washer;
attaching the barrier and the at least one barrier extender to the skin of the wearer such that the barrier ring is within the barrier such that the barrier ring forms a seal with the pouch such that urine can enter the pouch;
wrapping a cummerbund style belt about the wearer to support the pouch, the cummerbund style belt including a through hole with a diameter slightly less than the diameter of the washer, the pouch extending through the through hole and the washer not extending through the through hole; and
tightening and securing the cummerbund style belt such that the cummerbund style belt applies downward compression on the washer and towards the wearer's skin such that the washer, in turn, applies its downward compression on the barrier and the at least one barrier extender to secure the barrier and the at least one barrier extender against wearer's skin throughout the full 360° circumference of the barrier.

10. A method in accordance with claim 9, wherein the at least one barrier extender comprises three barrier extenders adapted and configured to be coupled to the barrier to extend the full circumference of the barrier.

11. A system in accordance with claim 10, wherein the three barrier extenders are adapted and configured to be coupled to the barrier to expand the area couplable to a wearer's skin.

12. A method in accordance with claim 10, further comprising applying a fourth barrier extender to the barrier to expand the area couplable to a wearer's skin.

13. A method in accordance with claim 11, further comprising applying a fifth barrier extender to couple the washer to the barrier or the at least one barrier extender.

14. A method in accordance with claim 9, wherein the washer comprises a plurality of individual washers.

15. A method in accordance with claim 14, further comprising securing the plurality of individual washers to one another.

16. A method in accordance with claim 14, wherein the plurality of individual washers includes three or more individual washers.

17. A method in accordance with claim 1, further comprising the wearer taking a daily supplement including both omega-3 fatty acids.

18. A method in accordance with claim 17 wherein the omega-3 fatty acids includes eicosapentaenoic acid and docosahexaenoic acid.

19. A method in accordance with claim 1, further comprising the wearer taking a daily supplement including turmeric extract.

20. A method in accordance with claim 1, further comprising the wearer taking a daily supplement including quercetin.
